(19) United States
(12) Patent Application Publication
(10) Pub. No.: US 2022/0199222 A1
(43) Pub. Date: Jun. 23, 2022

(54) METHOD AND APPARATUS TO PROVIDE NUTRITION QUALITY EVALUATION COMPREHENSION AND SELECTION

(71) Applicant: MILKMAN, INC., Birmingham, AL (US)

(72) Inventors: Xuan HUANG, Birmingham, AL (US); Alexander ZOTOV, Birmingham, AL (US)

(21) Appl. No.: 17/554,996

(22) Filed: Dec. 17, 2021

Related U.S. Application Data
(60) Provisional application No. 63/126,894, filed on Dec. 17, 2020.

(51) Int. Cl. G16H 20/60; G06V 20/68; G06V 20/62; G06F 16/2457

(52) U.S. Cl. CPC G16H 20/60; G06F 16/2457; G06V 20/62; G06V 20/68

(57) ABSTRACT

Methods, articles, and devices for evaluating the contribution of a food item(s), recipe(s), and meal plan to nutritional requirements are disclosed. Nutritional criteria for a person, which may be based upon general or specific dietary recommendations or customized for a particular individual's dietary needs, include reference value(s) or ranges for each of a plurality of nutrients, including vitamins, minerals, or food groups with a common profile. Indicia of satisfying the person's nutritional criteria are generated for a given food item, recipe, or meal plan provided to or selected by the user. The indicia include a plurality of bars arranged along a circumference of a circle, the length, direction and other aspects of each bar corresponding to the extent to which the one food item, recipe, or meal plan meets a nutritional criterion's reference value. In this way, an easily understood visual representation of the nutritional value of food choices is provided and well-informed dietary decisions can be made.

### PRIORITY CLAIM

This application claims priority to and the benefit of Provisional Patent Application No. 63/126,894, filed Dec. 17, 2020.

### BACKGROUND

In the U.S., adult obesity rate is currently 42.4% and more than half of all adults have at least one preventable chronic disease according to the Centers for Disease Control and Prevention (CDC). Obesity and many chronic diseases are directly caused by poor diets.

The unsettling reality is not due to a lack of dietary information and guidelines. The Health and Medicine Division of the National Academies of Sciences, Engineering, and Medicine (NASEM) has published the Dietary Reference Intakes (DRIs), which is a set of specific reference values for each nutrient to be met for different life stage groups. The U.S. Department of Agriculture (USDA) and the Department of Health and Human Services (DHHS) jointly publish Dietary Guidelines for Americans (DGAs) every 5 years since 1980. The Food and Drug Administration (FDA) mandates strict nutrition labeling regulations on most food products.

Despite the government's efforts, these nutrition advices are not well heeded. According to a survey conducted by NASEM, less than 10% of Americans consume a diet consistent with DGAs. And according to the USDA, 8 out of 10 Americans think advice about what to eat is conflicting.

The poor adherence of these guidelines is partially due to the fact that quantitative information is fragmented and tedious to follow and track. For example, the Dietary Reference Intakes issued by the Food and Nutrition Board of the Institute of Medicine, National Academy of Sciences (available at http://ods.od.nih.gov/HealthInformation/Dietary_Refeerece_Intakes.aspx) summarizes the DRIs in six tables and around 70 reference values in the form of lower or upper limits for a person to compare its diet to. It is almost impossible for an unsophisticated user to check how its daily consumption compares to these reference values, even if it has a way to track and add up its intake of each nutrient. The U.S. Food & Drug Administration issues requirements for food nutrition fact labeling (an example of which is available at https://www.fda.gov/food/new-nutrition-facts-label/how-understand-and-use-nutrition-facts-label). Though it is useful for selecting products, it is impractical to keep a record of and add up every food item one person consumes during a day, let alone comparing the summation of consumption with reference values.

For the abovementioned reasons, inventions that help users clearly and quickly evaluate nutrition quality of a food item, a recipe, and/or a meal plan are needed. Such inventions can facilitate making food choices, making or selecting recipes or meal plans, and therefore make it easier for users to lead a healthy lifestyle.

### SUMMARY

Disclosed herein is a method, article and apparatus to provide nutrition quality evaluation of a food item, and/or combination of food items that form a recipe or a meal plan and displaying that evaluation visually to a user. Embodiments of the present invention thus provide a nutritional instrument or aid for measuring and displaying a nutritional score of a food item, recipe, or meal plan. This can be personalized for each individual or can be used for a certain demographic that share similar nutrition needs.

One embodiment is a method of evaluating the contribution of at least one of a food item, a recipe, and a meal plan to nutritional requirements, which includes providing a database comprising a plurality of food items, a plurality of recipes, and a plurality of meal plans, wherein each meal plan comprises a plurality of recipes, each recipe comprises a plurality of food items, and each food item comprises a plurality of nutrient values; identifying nutritional criteria for a person comprising a reference value for each of a plurality of nutrients; and providing for at least one of a food item, recipe, or meal plan, indicia of satisfying the nutritional criteria comprising for each criterion within said nutritional criteria a bar having a length and an origin, the origins of the plurality of criteria arranged along a circumference of a circle, the length of each bar corresponding to a magnitude of the nutrient value of the at least one food item, recipe, or meal plan for that criterion relative to its reference value, the bar radiating away from a center of the circle if the nutrient value meets the reference value and radiating toward the center if the nutrient value does not meet its reference value. This method may also include receiving a selection of the at least one food item, recipe, or meal plan, for example, from a user using application software on a client device implementing the method.

In some embodiments, the method may also include providing a plurality of food items, recipes, or meal plans, and in response to a selection or deselection of at least one food item, recipe, or meal plan, updating the indicia to reflect the nutrient values of the selection or deselection. This allows a user to see the relative nutritional value of his or her choices. The nutritional criteria used may be for one of a plurality of generic profiles, each profile having a corresponding nutritional criteria, or it may be based upon input received from a user of personal and demographic information.

In a preferred embodiment, methods of the present invention utilize color, wherein the bar for each criterion is a particular, and nutritionally related criteria are the same color. In a still preferred embodiment, the color level may be saturated to correspond to a magnitude of the nutrient value of the at least one food item, recipe, or meal plan for a criterion relative to its reference value.

In one embodiment, the method further includes receiving an image of a recipe or food item and then providing the indicia based upon at least one food item in the database corresponding to the image. The image of the food item or of the recipe is matched to a food item or plurality of food items (in the case of a recipe) in the database. The image may also be of a grocery list of a plurality of food items. Accordingly, the nutritional value of the grocery list is displayed and any deficiencies may be identified.

The reference value for a nutritional criterion may be a range for some nutrients. In one embodiment, the reference value for a plurality of nutrients within the nutritional criteria comprises an upper threshold and a lower threshold, and the bar radiates away from the center if the nutrient value is between the upper and lower thresholds and the bar radiates towards the center if the nutrient value is not between the upper and lower thresholds.

Another embodiment of the present invention is a nutritional instrument or aid for providing readily understood nutritional value of at least one food item, recipe, or meal plan relative to nutritional criteria, comprising a substrate marked with indicia of a plurality of nutrient criteria, each criterion having an origin corresponding to a reference value for a nutrient, wherein the plurality of origins are arranged along a circumference of a circle, and wherein for each nutrient criterion, a bar is extended radially from its origin a length corresponding to a magnitude of its nutrient value of the at least one food item, recipe, or meal plan for that criterion relative to its reference value, the bar radiating away from a center of the circle if the nutrient value exceeds the reference value and radiating toward the center if the nutrient value is less than its reference value. The substrate may be selected from the group consisting of paper, packaging of a food item, and a display of a computing device. In a preferred embodiment, the bar for each criterion is a color, and nutritionally related criteria are the same color, and the saturation of a color for a criterion may correspond to a magnitude of the nutrient value of the at least one food item, recipe, or meal plan for that criterion relative to its reference value.

The reference value for a nutritional criterion may be a range for some nutrients. In one embodiment, the reference value for a plurality of nutrients within the nutritional criteria comprises an upper threshold and a lower threshold, and the bar radiates away from the center if the nutrient value is between the upper and lower thresholds and the bar radiates towards the center if the nutrient value is not between the upper and lower thresholds.

Another embodiment of the present invention is an device for evaluating the contribution of at least one of a food item, a recipe, and a meal plan to nutritional requirements, comprising a processor and a display, where the processor programmable to access a database comprising a plurality of food items, a plurality of recipes, and a plurality of meal plans, wherein each meal plan comprises a plurality of recipes, each recipe comprises a plurality of food items, and each food item comprises a plurality of nutrient values; identify nutritional criteria for a person comprising a reference value for each of a plurality of nutrients; and display on the display for at least one of a food item, recipe, or meal plan, indicia of satisfying the nutritional criteria comprising for each criterion within said nutritional criteria a bar having a length and an origin, the origins of the plurality of criteria arranged along a circumference of a circle, the length of each bar corresponding to a magnitude of the nutrient value of the at least one food item, recipe, or meal plan for that criterion relative to its reference value, the bar radiating away from a center of the circle if the nutrient value meets the reference value and radiating toward the center if the nutrient value does not meet its reference value. In a preferred embodiment, the device includes an image capture device, and the processor is programmable to receive an image of a food item or recipe from the image capture device and to provide the indicia based upon at least one food item in the database corresponding to the image.

### BRIEF DESCRIPTION OF THE DRAWINGS

The present invention will be explained, by way of example only, with reference to certain embodiments and the attached figures, in which:

FIG. 1 is a schematic drawing of a network and network-connected elements that may be utilized in one or more embodiments of the present invention.

FIG. 2 is a block diagram illustrating the key components of the application server and the database that may be used in one or more embodiments of the present invention.

FIG. 3A is an exemplary data structure of a recipe.

FIG. 3B is an exemplary data structure of a meal plan.

FIG. 3C is an exemplary data structure of nutrient values of a food item.

FIG. 3D is an exemplary data structure of nutrient values of a recipe.

FIG. 3E is an exemplary data structure of nutrient values of a meal plan.

FIG. 4A is a shinometer of an exemplary meal plan.

FIG. 4B is a shinometer of an exemplary recipe.

FIG. 4C is a shinometer of an exemplary meal plan personalized for an exemplary user.

FIG. 5A is an exemplary user profile.

FIG. 5B is an exemplary set of user nutrient reference values mapped from its profile.

FIG. 6 demonstrates how the shinometer can be used to inform users to make healthy food choices.

### DETAILED DESCRIPTION

In the following description, food items refer to fresh produce, processed food, meat, fish, poultry, dairy products, spices, and raw ingredients such as flour, sugar, and cooking oil, and also semi- or fully cooked meal/deli, wine, liquor, beer, and beverage. They are ingredients constituting recipes, and subsequently meal plans.

In the following description, nutrient value is the actual measured content of a nutrient in a food item/recipe/meal plan, where the nutrient can be that of traditional meaning such as the element Calcium (Ca), or can be a food group that shares similar nutrient profile, such as vegetables. Nutrient reference value is a lower or upper threshold value set by a diet regimen or guideline. For example, daily Calcium DRI for female 31-50 y.o. needs to be above 1,000 mg and below 2,500 mg. In another example, the DASH (Dietary approach to stop hypertension) diet recommends 4 to 5 servings of vegetables (veg) every day. In the following description, nutrient criterion refers to a logical expression between nutrient value and its reference values. For example, 1000<v_{Ca}<2500 or v_{veg}>4 where v represents the nutrient value and its subscript identifies the nutrient.

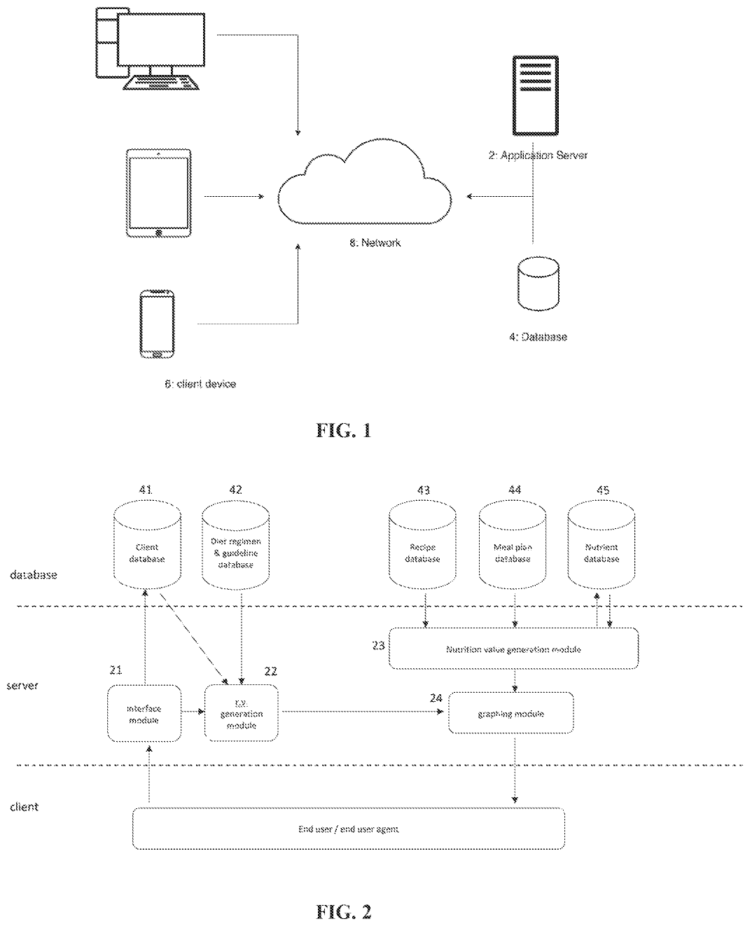

FIG. 1. is a schematic drawing of a network and network-connected elements in which embodiments of the present invention may be used. A network 8 connecting the application server 2, database 4, and the client device 6. Data may be provided through interaction with the client device 6 and/or extracted from the database 4. The application server 2 aggregates and analyzes data to produce the shinometer graph. The client device 6 may be used by the end user directly, or by its agent such as a dietitian or a physician.

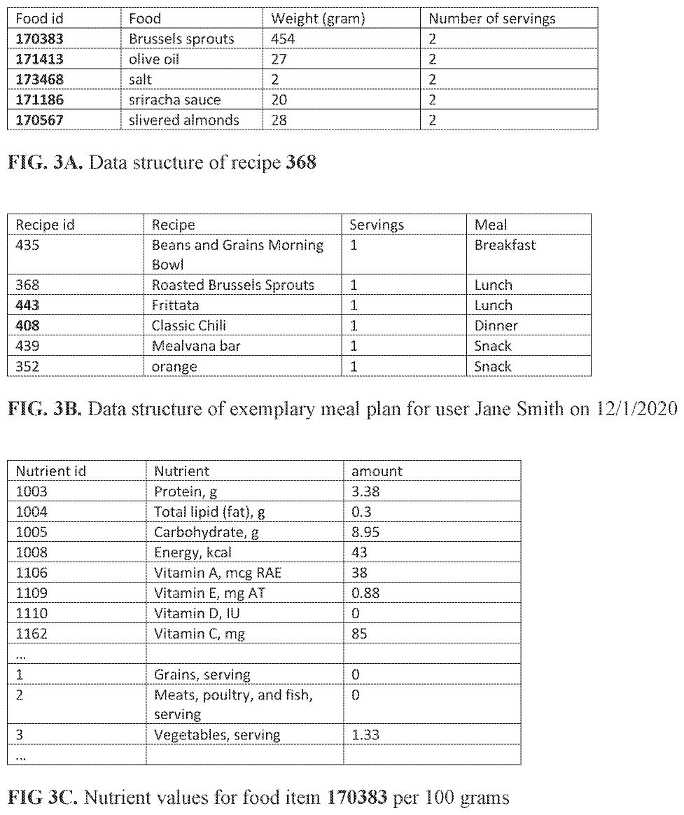

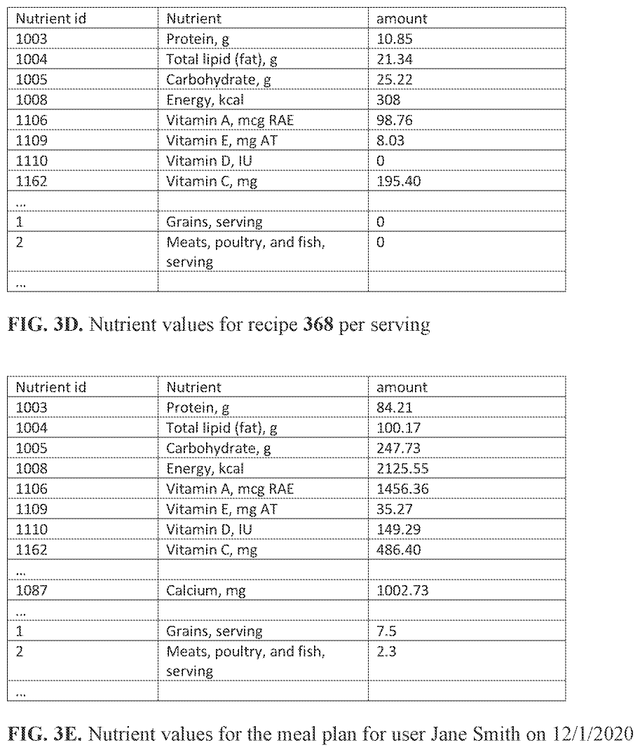

FIG. 2. is a block diagram illustrating the key components of the application server 2 and the database 4. The nutrient value generation module 23 aggregates data from the recipe database 43, the meal plan database 44, and the nutrient database 45 to extract or compute nutrient value for a food item or combination of food items such as a recipe or a meal plan. In an independent process, the end user or its agent provides input through an interface module 21 and may be stored in the user database 41. Input and stored user profile is then used to filter out relevant diet regimen & guidelines at the nutrient reference value generation module 22. Lastly, the graphing module 24 takes in the nutrient values and their corresponding reference values, computes a score for each nutrient criterion, and generates a shinometer graph that displays the scores. To illustrate the nutrient value generation module 23, one example is provided in FIG. 3. Food item Brussels Sprouts is an ingredient in the recipe Roasted Brussels Sprouts (recipe id: 368), shown in FIG. 3A. And recipe 368 is included in a meal plan for an imaginary user Jane Smith on the date Dec. 1, 2020, shown in FIG. 3B. FIG. 3C shows the nutrient values for Brussels Sprouts. Simple weighted summation is used by combining recipe data and nutrient values of each ingredient to produce nutrient values for the recipe, shown in FIG. 3D. Similarly, weighted summation is used by combining meal plan data and nutrient values of each recipe to produce nutrient values for the sample meal plan, shown in FIG. 3E. The abovementioned calculation is completed in the nutrient value generation module 23 and results may be stored in the nutrient database 45 for quick access.

Nutrient values of a recipe or a meal plan are forwarded to the graphing module 24. When no personalization is enabled, nutrient reference value generation module 22 can extract corresponding generic reference values and criteria from the diet regimen & guideline database 42 and forward them to the graphing module 24. For each criterion, a score is then calculated. In one embodiment, the score is calculated using the following formula:

min(tan h(log₂(UL/v)),tan h(log₂(v/LL)))

where UL is the upper limit of the criterion, if there is one, and the LL is the lower limit of the criterion, if there is one. v represents the nutrient value. In a nutshell, the formula returns a bounded positive value when the criterion is met and a bounded negative value when the criterion is not. The formula itself and the exact numeric values of these scores are not essential to the invention. However, it is preferred that the calculated scores reflect how the nutrient values meet the reference values. The scores' graphic representation in the shinometer that provides visual differentiation between meeting versus not meeting nutrient criteria is a key advantage and objective of preferred embodiments of the invention.

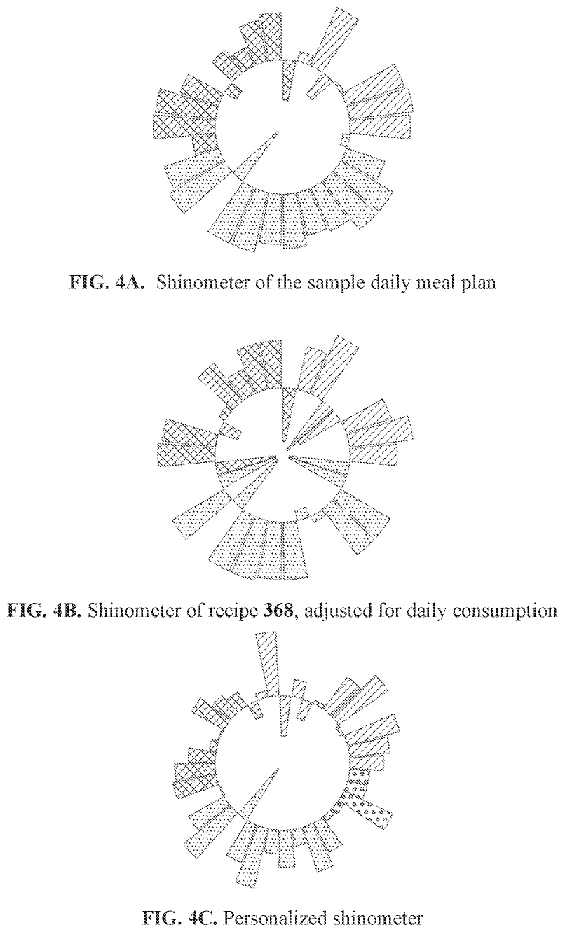

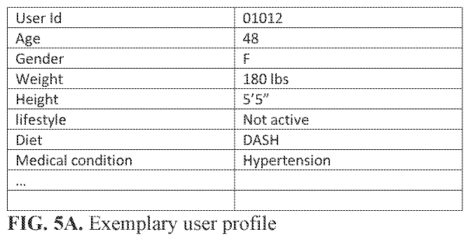

One example of generic reference values may be the Reference Daily Intakes (RDIs) which are set by the FDA for nutrition labeling. The RDI for Calcium is (at least) 1300 mg based on reference caloric intake of 2,000 calories for adults and children aged 4 years and older. For the sample meal plan in FIG. 3B, the nutrient value for Calcium is 1002.73 mg, shown in FIG. 3E. The Calcium nutrient score is then calculated to be −0.358, indicating Calcium criterion not met. In the similar fashion a score is calculated for every nutrient of concern. Scores are then plotted in a circular barplot. FIG. 4A is the shinometer displaying these scores. Each bar represents score of one nutrient. When the score is positive, the bar is pointing outward and when negative, pointing inward. The length of the bar may be proportional to the absolute value of the score. Colors may be used to identify the nutrient or nutrient group. In the example shown, red color is used to indicate macronutrients criteria, orange the elements, and yellow the vitamins. Further, each bar, especially the bars that point inward indicating a lack or overabundance of a nutrient can be labeled clearly what particular nutrients may need to be attended to. Color saturation may be used to further enhance the visual perception of positive versus negative values. In the example shown, negative bars are shown completely desaturated. The order in which the bars are arranged is not essential to the invention. The visual impact conveyed through collective display of all bars is the essence of a preferred embodiment of the invention.

Since a recipe or a single food item is only a portion of daily consumption, it is less meaningful to be compared to daily reference values directly. Their nutrient scores should be adjusted to better represent their proportional fulfillment of daily requirement. An example of adjustment method may be through the formula:

v_{adj}=v·C/c

where v represents the original nutrient value, v_{adj} represents the adjusted nutrient value, c represents the caloric value of the recipe or food item, and C represents the target daily calories. The adjusted nutrient values are then used to calculate the nutrient scores. For example, FIG. 4B is the shinometer displaying the adjusted nutrient scores for recipe 368. A single food item is seen in this description as a special case of recipes and a shinometer can be produced accordingly.

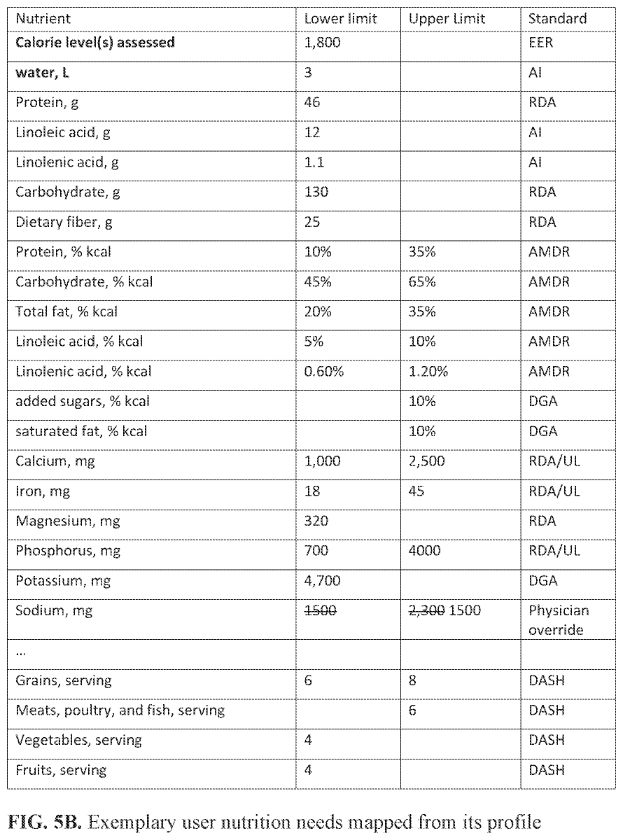

When personalization is called for, input and stored user data is used to filter out the relevant diet regimen & guidelines and generate more specific nutrient reference values and criteria. FIG. 5A provides an exemplar user profile and FIG. 5B the extracted nutrient reference values corresponding to her profile. A personalized nutrient criterion may also include quantifiable diet restrictions. For example, among the criteria shown in 5B, some are imposed by the DASH diet, the user's dietary choice. The nutrient value for the “Meats, poultry, and fish, serving” is 2.3 serving shown in FIG. 3E and its reference value is 6 as the upper limit shown in FIG. 5B. A score is calculated correspondingly to be 0.88, which indicates meeting this DASH criterion.

FIG. 4C shows the personalized shinometer of the sample meal plan for the imaginary user. In additional to the macronutrient, element, and vitamin groups, another group of crimson-colored bars are displaying how the meal plan is meeting her DASH diet criteria.

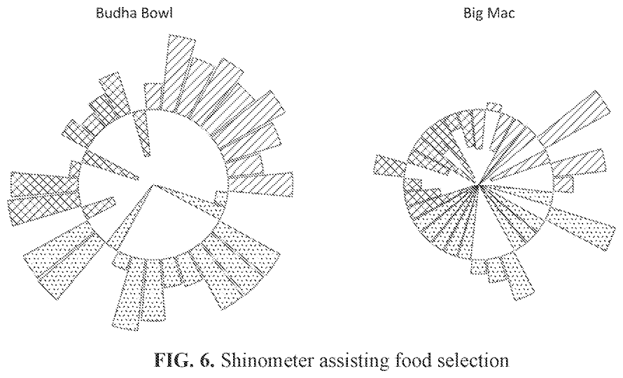

FIG. 6 demonstrates how effective shinometer helps food selection. A healthy food choice is expected to meet most nutrient criteria and results colorful bars pointing outward, resembling a shining sun. The tool enables an unsophisticated end user to quickly and clearly perceive the nutrient value of a food item/recipe/meal plan and how the nutrient values fits its diet and guidelines, so they can make healthy food choices.

In one embodiment, the interface module 21 includes an interactive configuration that displays an array of one or more of food items, recipes, or meal plans. Selections of food items, for example, are received and a shinometer is generated, as described herein, and displayed for the selected items. The user may then select and de-select additional food items, via the interface module 21, and the shinometer is updated accordingly in accordance with the methods described herein. This provides near instantaneous feedback to the user of the nutritional content and profile of various combinations of food and allows the construction of a balanced selection of food items. Similarly, an array of recipes or meal plans may be displayed in conjunction with (or separately from) the food items and a shinometer is dynamically updated as particular recipes or meal plans are selected, de-selected, or modified by user input. In these embodiments, the dynamically updated shinometer may be created with reference to the user's individual profile, to a more generic recommendation, or to a predetermined set of nutritional criteria or templates (such as the DASH Criteria or other dietary profile).

In another embodiment, recipes from any source are received by the nutrient value generation module 23 and a shinometer is generated for the recipe with respect to a selected (or predetermined) reference value from the nutrient reference value generation module 22, thus allowing an immediate evaluation and display of the nutritional score for the recipe. Recipes may be manually entered, received as data (text) files, ascertained and read from web pages, recognized from a hard copy (e.g., a cookbook or notecard) using image capture and processing (e.g. OCR) of a client device 6, such as a smart phone or tablet, or any other means of receiving recipe information. Similarly, embodiments of the present invention may receive a grocery list, or scan or receive data associated with purchase of groceries (i.e., food items), such as a data file from a point of sale or online check-out system or scanning a receipt, and generate a shinometer that displays the nutritional score of the shopping cart. This feature assists users to identify deficiencies in their food choices.

In a still further embodiment, image capture and processing capabilities of a client device 6 may be used to generate a shinometer from the existing food nutrition fact labels that appear on packaging of many food items. The image capture and processing capabilities also may be utilized to recognize food items that do not have a nutrition fact label, such as fresh produce or meats, and generate and display a shinometer. The image capture and processing capabilities also may be utilized to identify food items by barcodes or QR codes. For some devices, the weight (mass) of the food item may be estimated based upon three-dimensional data capture such as from LiDAR scanning to increase the accuracy of the nutrient value of the item. In embodiments of the invention utilizing this capability, a shinometer may be generated from the scanning, image capture, and processing of data from a plate of food, for example, to estimate the nutritional value of pre-prepared food, or food prepared by others. This provides a user an immediate nutritional evaluation and score of that food item with reference to the user's particular dietary or nutritional needs, or with reference to a generic or pre-selected dietary profile or set of criteria, including while shopping for, selecting food items, or dining out.

Although the present invention has been described and shown with reference to certain preferred embodiments thereof, other embodiments are possible. The foregoing description is therefore considered in all respects to be illustrative and not restrictive. Therefore, the present invention should be defined with reference to the claims and their equivalents, and the spirit and scope of the claims should not be limited to the description of the preferred embodiments contained herein.

1. A method of evaluating the contribution of at least one of a food item, a recipe, and a meal plan to nutritional requirements, comprising:
providing a database comprising a plurality of food items, a plurality of recipes, and a plurality of meal plans, wherein each meal plan comprises a plurality of recipes, each recipe comprises a plurality of food items, and each food item comprises a plurality of nutrient values;
identifying nutritional criteria for a person comprising a reference value for each of a plurality of nutrients;
providing for at least one of a food item, recipe, or meal plan, indicia of satisfying the nutritional criteria comprising for each criterion within said nutritional criteria a bar having a length and an origin, the origins of the plurality of criteria arranged along a circumference of a circle, the length of each bar corresponding to a magnitude of the nutrient value of the at least one food item, recipe, or meal plan for that criterion relative to its reference value, the bar radiating away from a center of the circle if the nutrient value meets the reference value and radiating toward the center if the nutrient value does not meet its reference value.

2. The method of claim 1, further comprising receiving a selection of the at least one food item, recipe, or meal plan.

3. The method of claim 2, further comprising providing a plurality of food items, recipes, or meal plans, and in response to a selection or deselection of at least one food item, recipe, or meal plan, updating the indicia to reflect the nutrient values of the selection or deselection.

4. The method of claim 3, wherein said person is selected from one of a plurality of generic profiles, each profile having a corresponding nutritional criteria.

5. The method of claim 3, further comprising before said identifying step, receiving input from a user of personal and demographic information, and wherein said identifying step comprises determining nutritional criteria for the user based on the input information.

6. The method of claim 1, wherein the bar for each criterion further comprises a color, and nutritionally related criteria are a same color.

7. The method of claim 6, wherein a saturation of a color for a criterion corresponds to a magnitude of the nutrient value of the at least one food item, recipe, or meal plan for that criterion relative to its reference value.

8. The method of claim 1, further comprising before said providing step, receiving an image of a recipe or food item and providing the indicia based upon at least one food item in the database corresponding to the image.

9. The method of claim 8, wherein the image comprises a grocery list of a plurality of food items.

10. The method of claim 1, wherein the reference value for a plurality of nutrients within the nutritional criteria comprises an upper threshold and a lower threshold, and the bar radiates away from the center if the nutrient value is between the upper and lower thresholds and the bar radiates towards the center if the nutrient value is not between the upper and lower thresholds.

11. A nutritional aid for providing readily understood nutritional value of at least one food item, recipe, or meal plan relative to nutritional criteria, comprising:
a substrate marked with indicia of a plurality of nutrient criteria, each criterion having an origin corresponding to a reference value for a nutrient, wherein the plurality of origins are arranged along a circumference of a circle, and wherein for each nutrient criterion, a bar is extended radially from its origin a length corresponding to a magnitude of its nutrient value of the at least one food item, recipe, or meal plan for that criterion relative to its reference value, the bar radiating away from a center of the circle if the nutrient value exceeds the reference value and radiating toward the center if the nutrient value is less than its reference value.

12. The nutritional aid of claim 11, wherein the substrate is selected from the group consisting of: paper, packaging of a food item, and a display of a computing device.

13. The nutritional aid of claim 11, wherein the bar for each criterion further comprises a color, and nutritionally related criteria are a same color.

14. The method of claim 13, wherein a saturation of a color for a criterion corresponds to a magnitude of the nutrient value of the at least one food item, recipe, or meal plan for that criterion relative to its reference value.

15. The nutritional aid of claim 11, wherein the reference value for a plurality of nutrients within the nutritional criteria comprises an upper threshold and a lower threshold, and the bar radiates away from the center if the nutrient value is between the upper and lower thresholds and the bar radiates towards the center if the nutrient value is not between the upper and lower thresholds.

16. An device for evaluating the contribution of at least one of a food item, a recipe, and a meal plan to nutritional requirements, comprising:
a processor and a display, said processor programmable to:
access a database comprising a plurality of food items, a plurality of recipes, and a plurality of meal plans, wherein each meal plan comprises a plurality of recipes, each recipe comprises a plurality of food items, and each food item comprises a plurality of nutrient values;
identify nutritional criteria for a person comprising a reference value for each of a plurality of nutrients;
display on the display for at least one of a food item, recipe, or meal plan, indicia of satisfying the nutritional criteria comprising for each criterion within said nutritional criteria a bar having a length and an origin, the origins of the plurality of criteria arranged along a circumference of a circle, the length of each bar corresponding to a magnitude of the nutrient value of the at least one food item, recipe, or meal plan for that criterion relative to its reference value, the bar radiating away from a center of the circle if the nutrient value meets the reference value and radiating toward the center if the nutrient value does not meet its reference value.

17. The device of claim 16, further comprising an image capture device, and wherein said processor is programmable to receive an image of a food item or recipe from the image capture device and to provide the indicia based upon at least one food item in the database corresponding to the image.
